(19) United States
(12) Patent Application Publication
(10) Pub. No.: US 2021/0315632 A1
(43) Pub. Date: Oct. 14, 2021

(54) ELECTROSURGICAL DEVICE WITH INTEGRATED NERVE MONITORING

(71) Applicant: GYRUS ACMI, INC. D/B/A OLYMPUS SURGICAL TECHNOLOGIES AMERICA, Westborough, MA (US)

(72) Inventors: Thomas J. Holman, Princeton, MN (US); Nikhil M. Murdeshwar, Maple Grove, MN (US); Jeffrey D. Holton, Stanchfield, MN (US)

(21) Appl. No.: 17/224,849

(22) Filed: Apr. 7, 2021

Related U.S. Application Data
(60) Provisional application No. 63/008,088, filed on Apr. 10, 2020.

(51) Int. Cl. A61B 18/14; A61B 18/12; A61B 90/30; A61B 90/00

(52) U.S. Cl. CPC A61B 18/14; A61B 18/1206; A61B 2018/00434; A61B 90/361; A61B 90/30

(57) ABSTRACT

Systems and techniques for nerve monitoring are described herein. The system can include a remote transmitter configured to send a stimulation signal to a nerve, and an electrosurgical device including an end effector configured to perform a surgical function in response to receiving a therapeutic signal from an electrosurgical generator. The system can also include a receiver configured to detect the stimulation signal, and in response to detecting the stimulation signal, determine a location of the nerve, and an in-vivo transmitter configured to emit a location signal in response to the determined location of the nerve.

### CROSS-REFERENCE TO RELATED APPLICATION

This application claims the benefit of priority to U.S. Provisional Patent Application Ser. No. 63/008,088, filed Apr. 10, 2020, the content of which is incorporated herein by reference in its entirety.

### TECHNICAL FIELD

The present disclosure relates to nerve monitoring.

### BACKGROUND

Many medical procedures, such as electrosurgery, involve activity that can affect nerves or nerve bundles. Electrosurgery involves various techniques that can be used during medical procedures, such as cutting, clamping, coagulating, desiccating, fulgurating, or the like, of biological tissue. During electrosurgery, signals can be generated by an electrosurgical generator and provided to the biological tissue through an electrosurgical device. The electrosurgical energy can be provided to tissue via an end effector of the electrosurgical device. The end effector can include, for example, a forceps or jaw members alone or in combination with a cutting element.

Different medical procedures can use different therapeutic signals to achieve results specific to these different medical procedures. Various electrical metrics of the electrotherapeutic signals provided to the biological tissue being treated can be used to characterize these electrotherapeutic signals. These electrical metrics can include: polarity (e.g., monopolar, bipolar), AC and/or DC, frequency, signal amplitude, attack and decay profiles, or the like. During the medical procedure, the end effector can be used in proximity to a nerve or a nerve bundle.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a nerve proximity detection system.

FIGS. 2A-2D illustrate an example of a surgical device and end effectors which can be coupled to the device.

FIG. 3 illustrates an example of a pad including an array of transmitting sensors which can be used to detect nerve activity and proximity.

FIG. 4 illustrates an example of a graph showing normal peak-to-peak of a receiver voltage output and increased peak-to-peak voltage output when the receiver is in proximity to a nerve.

FIG. 5 illustrates an example of a pulsed output transitioning to steady-state output when a receiver is in proximity to a nerve.

FIG. 6 illustrates an example of a method of detecting nerve proximity.

### DETAILED DESCRIPTION

This document describes, among other things, devices and methods for monitoring nerve activity and proximity to a nerve. The present inventors have identified a need for monitoring the location of, or proximity to, nerves before, during, or after a medical procedure, such as electrosurgery. Such monitoring can allow a surgeon to avoid contact, and thus risk causing damage to a nerve during surgery, or determine whether the nerve of a patient was affected, either positively or negatively as a result of the medical procedure.

Electrosurgery can involve manipulating biological tissue using an end effector of an electrosurgical device. The end effector can include one or more of: a jaw, a forceps, a conductive spatula, a j-hook, electrical pads, or the like. The end effector can be located at or near the distal tip of the electrosurgical device such as at the distal end of an elongated shaft extending outward from a handpiece.

Manipulating tissue can involve applying an electrotherapy signal such as to produce a desired change in the biological tissue of a surgical patient. Energy at the end effector can be used to modify the biological tissue through, for example, localized heating, desiccating the tissue, changing the tissue structure, or destroying tissue at the cellular level. Modifying tissue can be accomplished, for example, by electrical energy strikes (e.g., pulses, signals, or the like) emitted by the electrosurgical device at the end effector, by heating the end effector, or the like, alone or in combination with mechanical tissue manipulation such as grasping, cutting, or the like. Similarly, a medical procedure in which an electrosurgical device is used can have one or more electrotherapy phases, such as, a heating phase, a drying phase, a cauterizing phase, or the like.

An electrosurgical electrical signal can be generated by an electrosurgical generator. An electrosurgical generator can produce a variety of electrosurgical waveforms, which, in turn, can be applied to obtain corresponding tissue effects, such as those described above. The electrosurgical generator can be connected to a handpiece such that the electrical signal can then be passed from the handpiece to the end effector, such as via conductors extending along the shaft between the handpiece and the end effector.

During a medical procedure such as a small incision surgery (e.g., a thyroidectomy, neck surgery, spine surgery, or the like), or during a process of grasping, grabbing, or gripping a piece of tissue between the jaws of an electrosurgical forceps and applying electrosurgical energy such as a current, the tissue can be heated or otherwise modified (e.g., cut). This process can cause energy to be directed close to a nerve or a bundle of nerves of a patient and can cause undesired and inadvertent damage to the nerve or nerve bundle. This damage can include, for example, pain, sensitivity, numbness, loss of function, muscle atrophy, weakness, paralysis, chronic neuropathy, or the like.

The present inventors have recognized, among other things, that a signal can be transmitted through a nerve to allow a physician to determine the location of nerves in an area of a patient before operating on the patient, and by locating a receiver in, on, or near the end effector to receive the transmitted location signal, and provide the physician feedback of nerve location during the medical procedure. Furthermore, pairing a source of illumination, such as a fiber optic illumination source with a receiver (e.g., located at or near the end effector) can allow a physician to visualize the location, and/or track the path of the nerve. This combination can reduce, inhibit, restrict or otherwise lower false detection of a nerve, both false positive and false negative detections. In an example, a camera can also be used instead of or in addition to a fiber optic illumination source.

Such a system can enhance early nerve identification, enhance neural preservation by minimizing, restricting, limiting, or reducing trauma, damage, or the like to a nerve. Likewise, determining nerve activity and location prior to a medical procedure can also allow a physician to assess neural integrity before and/or after the procedure. For example, this can be accomplished by comparing the neural activity before the procedure with the neural activity following the procedure to determine whether any change (desired or undesired) in neural activity occurred as a result of the procedure.

In an example, the system can include a receiver configured to detect a stimulation signal from a nerve. This stimulation signal can be sent from a transmitter (e.g., a remote transmitter) which can be located separate from the receiver, such as an electromyogram (EMG) stimulator or other similar stimulator which can be placed, connected to, or otherwise attached to a patient, such as near a surgical site or near a nerve or nerve bundle to be monitored. The remote transmitter can be coupled to a function generator, an electrosurgical generator, or the like, configured to deliver a stimulation signal through the nerve via the remote transmitter. In an example, the remote transmitter can include a sensor array, such as an array of transmitters included within a pad which can be placed or located on a portion of a body of a patient (e.g., on the back of a patient)

The receiver, in response to detecting the stimulation signal, can then determine a location of the nerve, which can include monitoring a proximity to the nerve. The proximity to the nerve can be a distance between the receiver (e.g., the distal tip of the receiver) and the nerve. The proximity to the nerve can be determined by measuring an impedance. In an example, the proximity to the nerve can include a determination of a threshold distance between the receiver and the nerve.

The system can also include an in-vivo transmitter (e.g., a location transmitter) that can be communicatively coupled to the receiver. The in-vivo transmitter can emit, transmit, send, or the like, a location signal in response to a determination by the receiver of a location of the nerve. The in-vivo transmitter can include an electrode, such as an intraoperative neuromonitoring (IONM) electrode, or the like. The system can additionally or alternatively include a proximity indicator configured to provide an indication in response to the location signal. In an example, the indication can be a visual indication component, such as a light (e.g. an illuminated light-emitting diode (LED)) or a message on a graphical user interface (GUI) indicating the distance between the receiver and the nerve. In another example, multiple LEDs can be used to indicate the proximity of the receiver to the nerve.

In an example, an electrosurgical device can be a part of the system. For example, the system can include a medical device including an electrosurgical end effector which in response to receiving a therapeutic signal from an electrosurgical generator, can perform a surgical function. Surgical functions can include delivering a current to cut, heat, desiccate, or the like, a portion of tissue. The end effector can be located on a distal portion of the medical device, such as at a distal end of an elongated shaft. The receiver, configured to receive a location signal from a nerve, can be located near, on, or within the end effector, such as within one or more of the jaw members of a surgical forceps.

The in-vivo transmitter and the proximity indicator can also be included as a part of the electrosurgical device. The electrosurgical device can also include a camera on the distal portion, at or near the end effector to allow a surgeon to visualize a portion of the tissue without the need for using a scope, such as an endoscope, or the like. The end effector can also include an illumination source, such as a fiber optic light, or other similar illumination sources. The illumination source can illuminate in response to the indication provided by the proximity indicator. The illumination source can also be operated manually by a surgeon such as, for example, by initiating an actuation such as pushing a button, turning a dial, engaging a switch, or the like.

The system can also include a control circuit configured to communicate with the electrosurgical generator and the receiver. In an example, the control circuit can be located on or within the electrosurgical device, within the electrosurgical generator, or within a separate component. In an example, when the distance between the end effector and the nerve are below a threshold, the control circuit can be configured to cease, stop, cut off, disengage, or the like, the therapeutic signal from the electrosurgical generator, such that the end effector does not receive an active current or signal when it is an undesired distance from the nerve (e.g., below a minimum threshold distance or proximity).

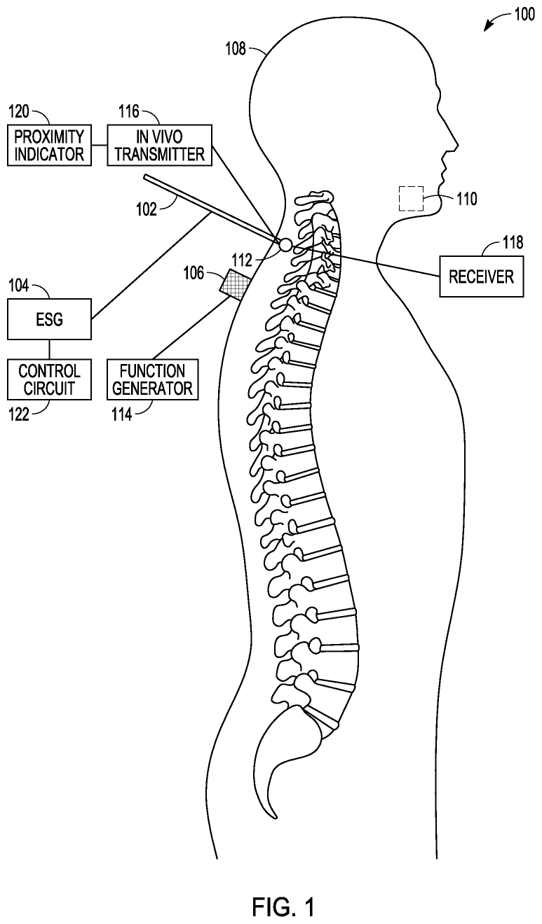

FIG. 1 illustrates an example of a nerve proximity detection system. In an example, the system 100 can include an electrosurgical generator 104 configured to send a therapeutic signal to a surgical device 102 such as an electrosurgical forceps, containing an end effector 112. The therapeutic signal can be a current or another similar energy to the end effector 112 (e.g., a forceps, jaws, electrode, cutting element, or the like) to perform a surgical function.

The system 100 can further include at least one remote transmitter 106, 110 which can be placed, located, or the like, on or within the body of a patient 108. The remote transmitter 106, 110 can include an electromyogram (EMG) stimulator, or other similar devices capable of transmitting a stimulation signal toward (e.g., in the direction of) a nerve or nerve bundle. In an example, the stimulation signal can penetrate the nerve, or otherwise be transmitted through, along, or the like, the nerve to allow a location of the nerve or proximity to the nerve to be detected, monitored, or the like. In an example a remote transmitter such as 106 can be located outside the body of the patient 108 (such as affixed, attached, or the like to the skin of the patient 108), or located within the patient 108 (e.g., inside the mouth of the patient 108) such as remote transmitter 110.

In an example, the remote transmitter 106, 110 can be coupled to a function generator 114, configured to send, transmit, or the like a stimulation signal (e.g., a voltage, current, or the like) to the remote transmitter 106, 110, which can then send, transmit, or the like, the stimulation signal to a nerve or nerve bundle. In an example, an electrosurgical generator 104 can generate an electrosurgical electrical signal. The electrosurgical generator 104 can produce a variety of electrosurgical waveforms, which, in turn, can be applied to obtain corresponding tissue effects. The electrosurgical generator 104 can be connected to the surgical device 102, such as connected to a handpiece of an electrosurgical device such that the electrical signal can then be passed from the handpiece of the surgical device 102 to the end effector 112, such as via conductors extending along a shaft between the handpiece and the end effector.

In an example, the remote transmitter 106, 110 can be used as “leave behind” markers. For example, the remote transmitter 106, 110 can be left on, within, or affixed to the patient 108 following a medical procedure to allow the nerve activity of the patient 108 to be monitored following the procedure. This can allow for a determination of whether the nerve activity of the patient changed or was otherwise affected, by the procedure. In an example, the remote transmitter 106, 110 may be placed on the patient 108 prior to surgery or other medical procedure to monitor nerve activity prior to surgery.

In an example, system 100 can include a receiver 118 configured to detect a stimulation signal from a nerve. The receiver 118 can be located at or near the end effector 112 of the surgical device 102. The receiver 118, in response to detecting the stimulation signal, can determine a location of the nerve. In an example, the receiver 118, and the remote transmitter 106, 110, can be configured to allow for bi-directional communication (e.g., allowing for a bi-directional signal to be transmitted) between them. This can include monitoring a proximity to the nerve such as a distance between the receiver 118 and the nerve (e.g., the distance between the distal tip of the receiver 118 and a point on the nerve, or the shortest distance between a point on the receiver 118 and the nerve, or the like). The proximity to the nerve can be determined by measuring an impedance, a voltage difference, or otherwise measuring the strength of the stimulation signal. For example, when the distance between the receiver 118 and the nerve is greater, the strength of the stimulation signal can be lower as compared to when the distance between the receiver 118 and the nerve is smaller. In an example, the measured impedance may be lower when the receiver 118 is closer to the nerve. For example, the impedance may be zero when the receiver 118 is in direct contact with the nerve and may increase as the receiver 118 is moved away from the nerve.

The system 100, can further include an in-vivo/location transmitter 116 configured to emit, transmit, send, or the like, a location signal in response to a determination by the receiver 118 of a location of the nerve in relation to the receiver or the end effector 112. The in-vivo transmitter 116, can be located at, on, near, or connected to the surgical device 102, such as either in a proximal handpiece portion or at, on, within, or near the end effector 112 or distal portion of the device 102. In an example, the in-vivo transmitter 116 can include an intraoperative neuromonitoring (IONM) electrode or other similar electrode or sensor which can be configured to send a location signal to a proximity indicator 120. The location signal can be based on the measured impedance. For example, as the receiver 118 moves in relation to the nerve, the impedance can be converted to an audio or light effect and sent (as the location signal) to a proximity indicator 120. In an example, the in-vivo transmitter can be further configured to emit a second stimulation signal (e.g., a square wave) to increase the resolution of the stimulation signal, which can provide an echolocation for the nerve or nerve bundle.

The proximity indicator 120 can be configured to provide an indication in response to receiving the location signal from the in-vivo transmitter 116. The proximity indicator 120, can be located on the surgical device 102, on the electrosurgical generator 104, on a display on a surgeon console, or the like. In an example, the indication can be visual such as a light emitted from a light-emitting diode (LED), or a message displayed on a graphical user interface (GUI) (e.g., indicating the distance between the receiver 118 and the nerve). In an example, multiple LEDs can be used to indicate the proximity of the receiver 118 to the nerve. For example, when the distance between the receiver 118 and the nerve is above a first/upper threshold value, an LED of a first color (e.g., green) can be illuminated. When the distance between the receiver 118 and the nerve is below a second/lower threshold value, an LED of a second color (e.g., red) can be illuminated. When the distance between the receiver 118 and the nerve is between the first and second threshold values, an LED of a third color (e.g., yellow) can be illuminated. In an example, the proximity indicator 120 can include an audio indication. For example, the proximity indicator 120 can include a speaker configured to emit an audio signal to provide the indication. The audio signal can be audible or can be an ultrasonic frequency. The thresholds thus can establish a series of zones which can be thought of a danger zone (e.g. below the second/lower threshold), a safe zone (e.g., above the first/upper threshold), and an intermediate zone (e.g., between the first and second thresholds).

In an example, the proximity indicator 120, can be located in, on, or near the surgical device 102, such as in a proximal or handpiece portion, or at or near the end effector 112. In an example, the proximity indicator 120 can be located separately from the surgical device 102, such as a graphical user interface (GUI) located separately from the device 102.

The system 100 can also include a control circuit 122 which can be included as a part of the electrosurgical generator 104 or located at or within the surgical device 102. The control circuit 122 can be configured to cease, stop, cut off, or otherwise disengage the therapeutic signal from the electrosurgical generator 104 to the end effector 112 when the distance between the receiver 118 and the nerve is below a threshold, such as the second/lower threshold as described above.

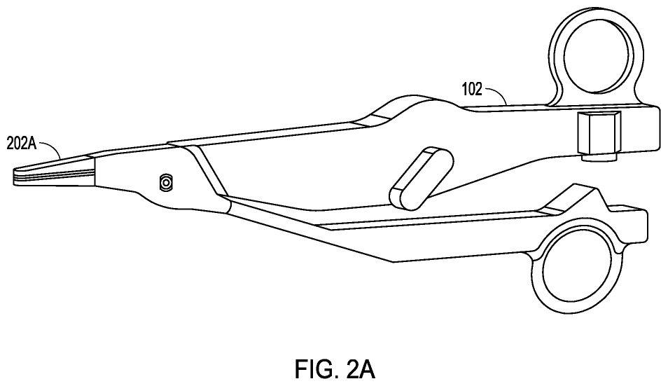

FIGS. 2A-2D illustrate an example of surgical device 102 as described in FIG. 1 above and end effectors such as 112 as described in FIG. 1 above which can be coupled to device 102. The device 102 can include a proximal portion which can include a handle and a distal portion which can include an end effector 202A such as a jaw or forceps. In the example of FIG. 2B, a jaw member 202B of an end effector 202A can include receiver 118, such as an embedded IONM electrode as described above for FIG. 1. In the example of FIG. 2C, a jaw member 202C of an end effector 202A can include a fiber optic illumination source 204. In an example, the indication as described above can be provided by the fiber optic illumination source 204 configured to illuminate in response to the proximity indicator 120 receiving the location signal. In an example, the fiber optic illumination source 204 can be configured to be controlled manually via a switch, button, or other similar actuators so as to illuminate a portion of tissue as desired, independent of receiving the location signal. In the example of FIG. 2D, a jaw member 202D of the end effector 202A can include a plurality of receivers 208-212 which can be the same or similar as 118 as described for FIG. 1 above. Returning to FIG. 2D, the plurality of receivers 208-212 can allow for a signal profile to be determined in which activity from multiple points of a nerve can be monitored by one or more of the plurality of receivers 208-212 spaced throughout the jaw member 202D.

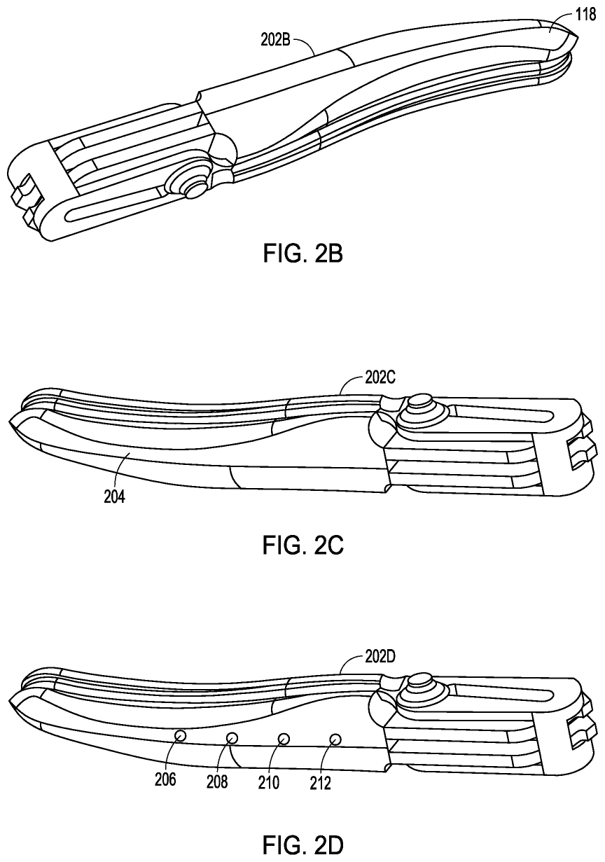

FIG. 3 illustrates an example of a pad including an array of transmitting sensors used to detect nerve activity and proximity. In an example, an array of transmitters 302-312, which can function as remote transmitters 106, 110 as described above for FIG. 1, can be located on a pad 300, patch, or other similar members which can be overlaid, placed, located or the like, on a patient 108, such as placed on the back of the patient 108. Such an array of transmitters 302-312 can form a two-dimensional sensor array, each of the transmitters 302-312 can have its own, separate, frequency which signal can be generated by the function generator 114.

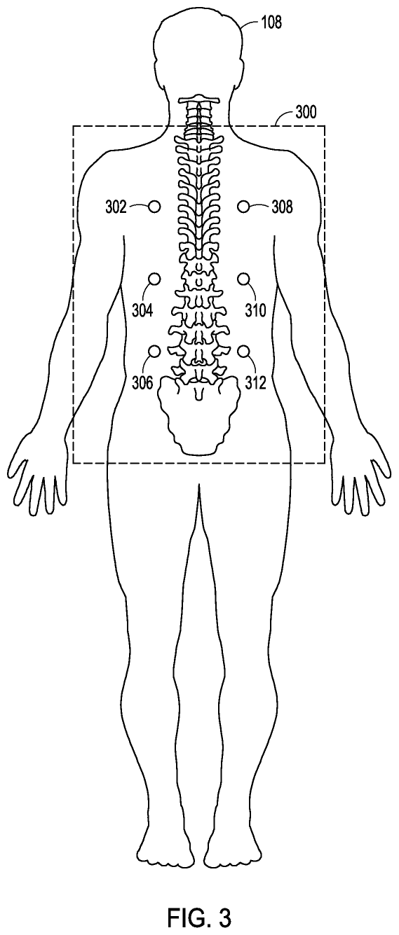

FIG. 4 illustrates an example of a graph showing normal peak-to-peak of a receiver voltage output and increased peak-to-peak voltage output when the receiver is in proximity to a nerve. In an example, the peak-to-peak voltage between the end effector 112 and the receiver 118 can be measured and related to the measured impedance. The solid curve 404 can represent the “normal” voltage difference between the end effector 112 and the receiver 118, such as when not in the presence of a nerve. When the receiver moves closer to a nerve, the measured impedance can decrease as the distance decreases (as described above for FIG. 1) and cause the peak to peak voltage to increase, as shown by the dotted curve 404. In an example, instead of measuring a voltage, impedance can be similarly measured and graphed.

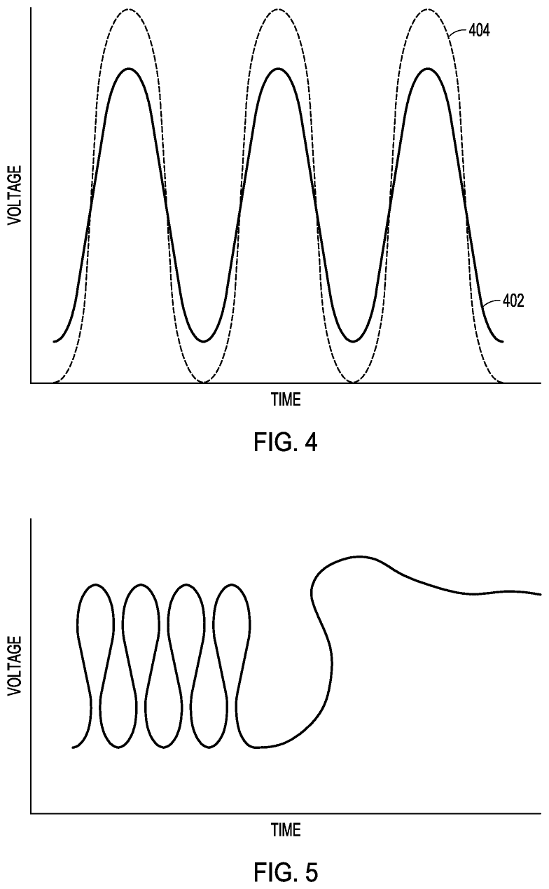

FIG. 5 illustrates an example of a pulsed output transitioning to steady-state output when a receiver is in proximity to a nerve. In an example, the second stimulation signal as described above can be transmitted as a sinusoidal or other pulsed wave. As the nerve is tracked and when the receiver 118 moves closer to a nerve, the pulsed waveform can be manipulated or changed, such as to switch to a steady output. In an example, the second stimulation signal can be a steady current, such as a steady DC wave, that can change to a pulsed waveform (e.g., a sinusoidal wave, a square wave, or a triangular wave) as the impedance decreases when the receiver moves closer to a nerve.

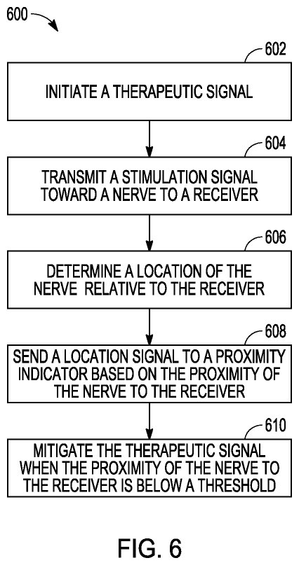

FIG. 6 illustrates an example of a technique, such as a method of detecting nerve proximity. At 602, a therapeutic signal can be initiated. The signal can be generated, for example via an electrosurgical generator such as 104 and transmitted to an end effector such as 112 to perform a surgical technique as described in FIG. 1 above. Returning to FIG. 6, operation 602 can be included in a method which includes use of an electrosurgical device such as 102, however, there can be variations of method 600 when this step is omitted. Such as, for example, monitoring nerve activity of a patient independent of, separate from, or the like, a surgical procedure. Such as, for example, monitoring nerve activity over a period of time to detect changes in the nerve activity of a patient, or monitoring nerve activity before, after, but not during, a surgical procedure.

Operation 604 can include transmitting a stimulation signal through a nerve to a receiver. This can include using a remote transmitter such as an EMG stimulator or other similar stimulating member such as an electrode to send, emit, transmit, or the like, a stimulation signal, via a function generator, to a nerve. The remote transmitter can be configured to send one or more of: a radio frequency (RF) signal, and infrared (IR) signal, an ultrasonic (US) signal, or an optical signal. The stimulation signal can include a direct current steady signal, a pulsed waveform such as a sinusoidal wave, a square wave, a triangular wave, or the like. In an example, the stimulation signal can be manipulated, modified, or the like to change when the receiver moves in relation to a location of the nerve. For example, when the receiver is a first distance away from the nerve, which can be an upper threshold limit, the stimulation signal can be a pulsed waveform. Then, when the receiver moves closer to the nerve, it can be manipulated to transform to a steady output.

Operation 606 can include determining a location of the nerve relative to the receiver. This can be performed by measuring an impedance, and the effect on a voltage difference between the receiver and an end effector of a medical device such as an electrosurgical device. For example, as the receiver moves closer to the nerve a measured impedance can decrease, causing a peak-to-peak voltage difference between the end effector and the receiver to increase, indicating a proximity of the receiver to the nerve. Determining the location of the nerve can include monitoring a proximity to the nerve, such that a user (e.g., a surgeon) can monitor, in real-time, the location of the receiver relative to the nerve and move the receiver as desired based on the proximity.

Operation 608 can include sending a location signal to a proximity indicator, based on the proximity of the receiver to the nerve determined at 606. The location signal can include converting the impedance measured at 606 to an audio or visual signal to send to a proximity indicator. For example, an upper threshold and a lower threshold distance between the receiver and the nerve can be determined. When the receiver is below the lower threshold distance, an audible signal can be emitted, such as through a speaker to alert a user that the receiver is below a desired distance to the nerve. In an example, the indication can be visual. For example, the location signal can include sending a message to a proximity indicator which is a graphical user interface (GUI), which can display the distance between the receiver and the nerve. Similarly, the visual indicator can include illuminating one or more LEDs. For example, when the distance is below a first desired distance an LED of a first color (e.g., red) can illuminate. Likewise, when the distance is above a second desired distance an LED of a second color (e.g., green) can illuminate. And, when the distance is between the first distance and the second distance an LED of a third color (e.g., yellow) can be illuminated. This can include illuminating a single LED capable of displaying multiple colors, or multiple different colored LEDs.

The visual indication can also include illuminating a fiber optic illumination source which can be located in the end effector of an electrosurgical device. The fiber optic illumination source can be configured to illuminate when the distance between the receiver and/or end effector is below the first desired distance to allow a user of the electrosurgical device to visualize the area of tissue being affected.

The method can include operation 610 which can include mitigating (e.g, reducing, limiting, ceasing, stopping, terminating, disengaging, or the like) the therapeutic signal initiated at 602 when the proximity to the nerve is below a distance threshold. For example, the first desired distance described above. In an example, operation 610 can include mitigating the therapeutic signal when the receiver detects, measures, or the like, the stimulation signal above a threshold value. In an example, a current or other therapeutic signal transmitted from an electrosurgical generator to an end effector of an electrosurgical device, causing the device to perform a surgical function (e.g., cutting, heating, desiccating, or the like a portion of tissue) can be stopped or cut off when the end effector is below a desired distance to a nerve or nerve bundle. This can prevent or limit the nerve or nerve bundle being damaged or otherwise affected in an undesired manner by the active end effector. As with operation 602, there can be variations of the method 600 when 610 is omitted, such as when an electrosurgical end effector is not being used as a part of the method 600.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A nerve proximity detection system comprising:
a remote transmitter configured to send a stimulation signal toward a nerve; and
an electrosurgical device including:
an end effector configured to, in response to receiving a therapeutic signal from an electrosurgical generator, perform a surgical function;
a receiver configured to:
detect the stimulation signal; and
in response to detecting the stimulation signal, determine a location of the nerve;
an in-vivo transmitter configured to emit a location signal in response to the determined location of the nerve.

2. The system of claim 1, wherein the electrosurgical device further comprises:
a proximity indicator configured to provide an indication in response to the location signal; and
a camera at a distal portion of the device.

3. The system of claim 1, wherein the remote transmitter and the receiver are configured to transmit a bi-directional signal between them.

4. The system of claim 2, wherein the proximity indicator includes a speaker configured to emit an audio signal to provide the indication.

5. The system of claim 2, wherein the proximity indicator includes a visual indication component to provide the indication.

6. The system of claim 2, wherein the end effector further includes an illumination source.

7. The system of claim 6, wherein the illumination source includes a fiber optic illumination source configured to illuminate in response to the indication provided by the proximity indicator.

8. The system of claim 2, wherein the in-vivo transmitter is communicatively coupled to the receiver, and in response to a determination of the location of the nerve is configured to send the location signal to the proximity indicator.

9. The system of claim 1, wherein to determine a location of the nerve includes:
to monitor a proximity to the nerve.

10. The system of claim 1, wherein the receiver is located on or within the end effector.

11. The system of claim 1, wherein to determine the location of the nerve includes to determine a proximity of the nerve to the end effector.

12. The system of claim 1 further comprising:
a control circuit configured to communicate with the electrosurgical generator and the receiver.

13. The system of claim 12, wherein when a distance between the end effector and the nerve is below a threshold, the control circuit is configured to mitigate the therapeutic signal from the electrosurgical generator.

14. The system of claim 12, wherein when the stimulation signal detected by the receiver is above a threshold, the control circuit is configured to mitigate the therapeutic signal from the electrosurgical generator.

15. The system of claim 1, wherein the proximity to the nerve is determined by measuring an impedance, wherein the in-vivo transmitter includes an intraoperative neuromonitoring (IONM) electrode, wherein the remote transmitter includes a sensor array and wherein the in-vivo transmitter is configured to send the location signal to a graphical user interface (GUI).

16. A method of detecting a location of a nerve, the method comprising:
transmitting a stimulation signal toward the nerve to a receiver;
determining a location of the nerve to the receiver based on the transmitted stimulation signal received by the receiver; and
sending the location signal to a proximity indicator based on the location of the nerve to the receiver.

17. The method of claim 16, wherein determining a location of the nerve to the receiver includes determining a proximity of the nerve to the receiver.

18. The method of claim 16, further comprising:
initiating a therapeutic signal to an electrosurgical end effector, the therapeutic signal configured to perform a surgical function.

19. The method of claim 18, further comprising:
mitigating the therapeutic signal when the proximity of the nerve to the electrosurgical end effector is below a threshold; and
transmitting the location signal to a graphical user interface (GUI).

20. The method of claim 16, wherein a transmitter is configured to send one or more of: a radio frequency (RF) signal, and infrared (IR) signal, an ultrasonic (US) signal, or an optical signal.

21. A nerve proximity detector comprising:
a receiver configured to detect a stimulation signal toward a nerve, and in response to a detection of the stimulation signal, determine a location of the nerve, wherein the location of the nerve includes a proximity of the receiver to the nerve, and wherein to determine a location of the nerve includes to monitor the proximity of the receiver to the nerve;
an in-vivo transmitter communicatively coupled to the receiver configured to emit a location signal in response to the determination of the location of the nerve;
a proximity indicator configured to provide an indication in response to the location signal from the in-vivo transmitter, wherein the proximity indicator includes at least one of: a visual indicator or an audible signal; and
wherein the proximity to the nerve is determined by measuring an impedance, and wherein the proximity to a nerve includes a determination of a threshold distance from the nerve.

22. The nerve proximity detector of claim 21, wherein the nerve proximity detector further comprises a control circuit configured to communicate with an electrosurgical generator and the receiver, wherein the nerve proximity detector includes at least one of: an illumination source or a camera, wherein the stimulation signal is sent toward the nerve from a remote transmitter, and wherein the remote transmitter and the receiver are configured to transmit a bi-directional signal between them.
